SEQUENCE LISTING (10 sequences)

SEQ ID NO: 1 - DNA, 6091 bp, Homo sapiens
  CDS (10)..(6054)
gatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcatacacaactgaatttgtggacctgggcaatgtctcagccttacgcaccttccgagtcctccgggccctgaaaactatatcagtcatttcagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccgcacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacatcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaag

SEQ ID NO: 2 - protein, 2015 aa, Homo sapiens [sequence omitted]

SEQ ID NO: 3 - DNA, 22 bp, Artificial Sequence
  Description of Artificial Sequence primer
gatgagaagatggcaaacttcc

SEQ ID NO: 4 - DNA, 22 bp, Artificial Sequence
  Description of Artificial Sequence primer
gctctggatccccgggggtgcc

SEQ ID NO: 5 - DNA, 20 bp, Artificial Sequence
  Description of Artificial Sequence primer
cacccccggggatccagagc

SEQ ID NO: 6 - DNA, 19 bp, Artificial Sequence
  Description of Artificial Sequence primer
ttcagtgtgtcctggccag

SEQ ID NO: 7 - DNA, 33 bp, Artificial Sequence
  Description of Artificial Sequence primer
ttcctcatcgtggttaacctgtacattgccatc

SEQ ID NO: 8 - DNA, 20 bp, Artificial Sequence
  Description of Artificial Sequence primer
ggagatgatgatgtaggtgg

SEQ ID NO: 9 - DNA, 25 bp, Artificial Sequence
  Description of Artificial Sequence primer
cgagagccaccgcgcatcactgctg

SEQ ID NO: 10 - DNA, 23 bp, Artificial Sequence
  Description of Artificial Sequence primer
ctctcccccgctgtgctgttttc

(19) United States
(12) Patent Application Publication
(10) Pub. No.: US 2003/0157600 A1
(43) Pub. Date: Aug. 21, 2003

(54) Sodium channel alpha subunits

(76) Inventors: Jonathan C. Makielski, Fitchburg, WI (US); Bin Ye, Forsyth, IL (US)

Correspondence Address: Bennett J. Berson, Quarles & Brady LLP, 1 South Pinckney Street, P O Box 2113, Madison, WI, 53701-2113, US

(21) Appl. No.: 10/077,054

(22) Filed: Feb. 12, 2002

(51) Int. Cl. C12Q 1/68; G01N 33/53; G01N 33/567; C07H 21/04; C12P 21/02; C12N 5/06; C07K 14/47

(52) U.S. Cl. 435/69.1; 435/320.1; 435/325; 530/350; 536/23.5; 435/6; 435/7.2

(57) ABSTRACT

The present invention discloses the cDNA sequence and the expressed amino acid sequence of a sodium channel α subunit, termed hH1b. A specific mutation in hH1b has been shown to display a different phenotype in relation to a human heart disease than other known human sodium channel α subunits with corresponding mutations. The present inventions provides new tools to design or identify new diagnostic and treatment strategies or agents for sodium channel related diseases or conditions.

### CROSS-REFERENCE TO RELATED APPLICATIONS

Not applicable.

### STATEMENT REGARDING FEDERALLY SPONSORED RESEARCH OR DEVELOPMENT

This invention was made with United States government support awarded by the following agency: NIH, Grant No. HL-56441. The United States has certain rights in this invention.

### BACKGROUND OF THE INVENTION

Sodium channel proteins embedded in cellular membranes of muscle cells, neurons and other excitable cells help produce and propagate electrical impulses and are implicated in many human diseases and conditions. Sodium channels are often composed of a pore-forming α subunit, having four homologous domains DI-DIV and six transmembrane regions S1-S6 per domain, and two auxiliary subunits β1 and β2. The α subunit is sufficient to form a functional channel for generating sodium current flow across cellular membranes.

Human cardiac sodium channels play a critical role in cardiac excitation. hNaᵥ1.5, a human cardiac sodium channel α subunit encoded by the SCN5A gene forms a functioning monomeric sodium channel that carries the inward Na current (I_{Na}) in the heart. The I_{Na} current is vital for excitation and conduction in working myocardium and in specialized conduction tissue such as Purkinje fibers.

Two distinct full-length polymorphic SCN5A clones (designated SCN5A hH1 and SCN5A hH1a, or simply hH1 and hH1a, respectively) that encode the hNaV1.5 human cardiac sodium channel have been isolated from human cardiac cDNA libraries (Gellens, M. E. et al., “Primary structure and functional expression of the human cardiac tetrodotoxin-insensitive voltage-dependent sodium channel,” Proc.Natl.Acad.Sci. U.S.A. 89, 554-558 (1992); Hartmann, H. A. et al., Effects of III-IV linker mutations on human heart Na⁺ channel inactivation gating. Circ.Res. 75, 114-122 (1994)). The significance of polymorphisms in the sodium channel is still unknown. For example, it is not known how such polymorphisms affect the mutation phenotype of SCN5A. Nonetheless, identified polymorphisms can help identify disease-associated mutations in SCN5A. For example, various SCN5A mutations are associated with congenital Long QT syndrome, idiopathic ventricular fibrillation and the Brugada syndrome (Keating and Sanguinetti 2001).

The reported sequences for hH1 and hH1a differ by nine amino acids with hH1 containing one additional amino acid (Q1077). However, the applicants independently sequenced both SCN5A forms again, thereby confirming the sequence of hH1a, while noting that the re-sequenced hH1 differed from the published hH1 sequence at seven amino acid positions, where each difference is, in fact, identical to the hH1a sequence. This change in the published sequence reduces the difference between hH1 and hH1a to just 3 amino acids (T559 vs. A559, Q1027 vs. R1027, and Q1077 vs. Q1077del (hH1 vs. hH1a, respectively) over a total length of 2016/2015 amino acids, respectively. All of the differences are confined to the cytoplasmic linkers between DI-II and between DII-III. Both hH1 and hH1a contain a histidine residue at amino acid 558 (H558), the site of a histidine-to-arginine polymorphism (H558R) (Iwasa, et al., “Twenty single nucleotide polymorphisms (SNPs) and their allelic frequencies in four genes that are responsible for familial long QT syndrome in the Japanese population,” J.Hum.Genet. 45, 182-183). The amino acid numbering follows that of the original hH1 clone which contains 2016 amino acids. In separate studies, the two known polymorphic forms showed only minor kinetic differences that can be attributed to different expression systems and study techniques including solutions, temperature, and protocols. (Gellens, M. E. et al., supra; Hartmann et. al., supra; and Wattanasirichaigoon et. al. 1999). Subtle differences in kinetics such as decay rates, inactivation midpoints, and late I_{Na}, however, may be important in controlling repolarization.

Sodium channel α subunits encoded by an SCN5A hH1a clone carrying an arrhythmogenic missense methionine-to-leucine mutation at amino acid 1766 (M1766L) further exhibit a significant inward sodium current level drop, relative to the current level in channels encoded by a wild type hH1a clone. Recently, M1766L in the hH1a background was shown to have a trafficking defect and to cause QT prolongation and ventricular arrhythmia (Valdivia et. al. 2001). These conditions can be rescued by low temperature, antiarrhythmic drug and β1 subunit (Valdivia et. al. 2001).

In another aspect, drugs that can alter sodium channel activities can relieve or prevent symptoms of certain conditions such as cardiac arrhythmias. Cardiac arrhythmias are abnormalities in the rate, regularity, or site of origin of the cardiac impulse, or a disturbance in conduction of the impulse that alters the normal sequence of atrial or ventricular activation. One known way to treat cardiac arrhythmias is to block the activity of a cardiac sodium channel. Sodium channel blockers used to treat cardiac arrhythmias include: Quinidine, Lidocaine, Procainamide, Mexiletine, Flecainide, Moricizine, and Disopyramide. Identifying other polymorphic forms of human cardiac sodium channel will advance our understanding of sodium channel-related heart problems and provide new tools for developing diagnostic, prophylactic and therapeutic strategies.

### BRIEF SUMMARY OF THE INVENTION

The present invention discloses a novel polymorphism in the hNaV1.5 sodium channel α subunit polypeptide, designated SCN5A hH1b or simply hH1b, as well as a nucleic acid molecule encoding same. A specific mutation in hH1b displays a different phenotype in relation to a human heart disease than other known human sodium channel α subunits carrying a corresponding mutation.

The present invention also includes various related nucleic acid molecules and polypeptides that are useful in various applications such as detecting the subunit and generating antibodies to the subunit. The present invention also relates to cloning and expression vectors and host cells containing same. In addition, the present invention includes methods for screening for an agent for altering (increasing or reducing) sodium channel activities. Furthermore, methods of using the nucleic acids and polypeptides to detect hH1b and generate antibodies to detect and purify hH1b are also included in the present invention. New diagnostic and treatment strategies for various sodium channel-related diseases and conditions are also enabled by the present invention.

It is an object of the present invention to identify a new polymorphic form of a human cardiac sodium channel.

It is another object of the present invention to provide new tools for designing diagnostic and treatment strategies for sodium channel related diseases and conditions.

Other objects, advantages and features of the present invention will become apparent from the following specifications and claims.

### BRIEF DESCRIPTION OF THE SEVERAL VIEWS OF THE DRAWINGS

FIG. 1 depicts the structure of the hNaV1.5 sodium channel α subunit polypeptide including amino acid variations among hH1, hH1a, and hH1b.

FIG. 2 shows whole cell I_{Na} for hH1, hH1a, and hH1b.

FIG. 3 shows current magnitude for wild type and hH1, hH1a, and hH1b clones and for each clone containing the M1766L arrhythmia mutation.

FIG. 4 summarizes the data of FIG. 3 as relative I_{Na} density.

### DETAILED DESCRIPTION OF THE INVENTION

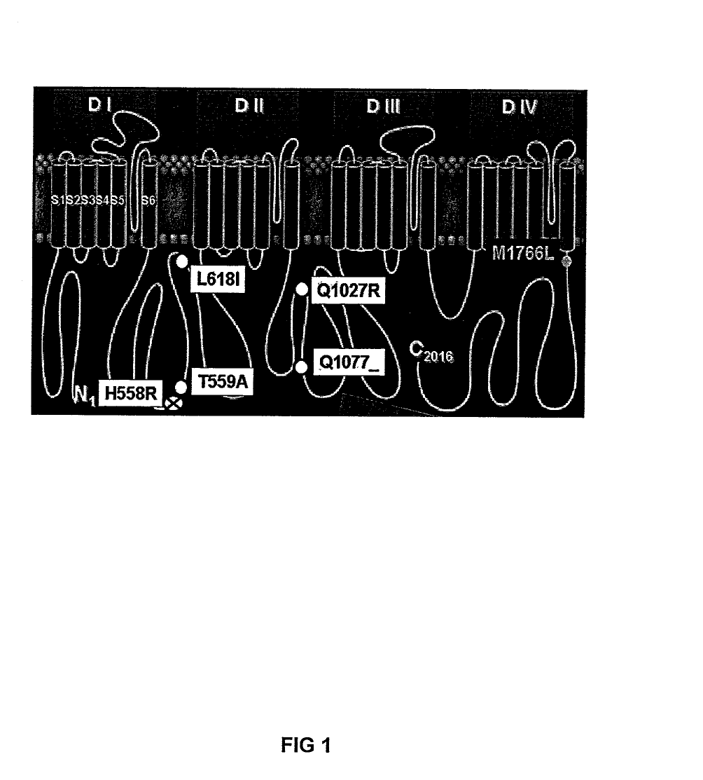

The present invention relates to a new variant form of the hNaᵥ1.5 sodium channel α subunit, designated SCN5A hH1b. A polynucleotide sequence that encodes SCN5A hH1b is attached as SEQ ID NO: 1. The amino acid sequence of the polypeptide encoded by SCN5A hH1b is attached as SEQ ID NO:2. The new clone differs from SCN5A hH1 by just four amino acids in the cytoplasmic linkers between DI-DII and DII-DIII (R558 vs. H558, 1618 vs. L618, R1027 vs. Q1027, and Q1077del vs. Q1077 (hH1b vs. hH1, respectively)). SCN5A hH1b differs from SCN5A hH1a by just three amino acids in the cytoplasmic linker between DI-DII (R558 vs. H558, T559 vs. A559, and I618 vs. L618 (hH1b vs. hH1a respectively). The only two amino acid positions at which hH1b differs from both hH1 and hH1a are amino acids 558 and 618. Table 1 summarizes the differences among the three hNaᵥ1.5 clones and from a sequence found in the Celera® human genome database. BLAST searching of the Celera human genome sequence showed only 2 differences between hH1b and the Celera sequence (R558 vs. H558 and I618 vs. L618), both of which are in the cyotplasmic linker between DI-DII. FIG. 1 also depicts the set of relevant mutations in the various clones, as well as the M1766L mutation.

TABLE 1
Deduced amino acid sequence comparisons for SCN5A clones.

| AA# | hH1 | HH1a | hH1b | ® Celera |
|---|---|---|---|---|
| 558 | H | H | R | H |
| 559 | T | A | T | T |
| 618 | L | L | I | L |
| 1027 | Q | R | R | R |
| 1077 | Q | . | . | . |

The hH1, hH1a, and hH1b sodium channel α subunits exhibits similar activation, inactivation and recovery kinetics when expressed in a cell line and, accordingly, hH1b provides an additional target for diagnosing and treating sodium channel-related diseases or conditions or for determining an agent's effects on sodium ion channels. More interestingly, hH1b differs from hH1 and hH1a in that the sodium channel current of the hH1b form is not lost in the presence of the M1766L mutation, as it is in the case of the hH1 and hH1a forms of the SCN5A gene. Because M1766L is associated with long QT syndrome in subjects that carry the hH1a form, the present invention further enables a test for determining the form of SCN5A carried by a subject, thereby facilitating assessment of the subject's risk for developing long QT syndrome or other condition.

The nucleic acid and amino acid sequences of hH1b disclosed herein enable the skilled artisan to produce diagnostic, prevention and treatment tools for sodium channel-related diseases including probes or hH1b specific antibodies for detecting hH1b expression in a biological sample.

In one aspect, the present invention relates to an isolated polypeptide comprising the amino acid sequence of SEQ ID NO: 2. An isolated polypeptide, as used herein, is one either synthetically derived or removed from its natural environment. An isolated polypeptide identical to SEQ ID NO: 2 carrying a substitution at one or more non-critical amino acid positions, where the substitution does not materially affect the function of the polypeptide. A non-critical amino acid position is an amino acid at a position other than those disclosed for hH1b in Table 1. Furthermore, an isolated polypeptide of the invention can also include any of the foregoing polypeptides having one or more amino acids at either or both of the N-terminus and C-terminus, where the additional amino acid(s) do not materially affect the function of the polypeptide. A change does not materially affect the function if the protein retains kinetic parameters within the ranges of those disclosed for hH1b in Table 2 and if the protein retains the ability to rescue the M1766L mutation, as described above. Any additional amino acids can, but need not, have advantageous use in purifying, detecting, or stabilizing the polypeptide. Likewise, small deletions or other rearrangements in the polypeptide that do not affect the function of the polypeptide are also within the scope of the invention. Such deletions are preferably deletions of fewer than 100 amino acids, more preferably of fewer than 50 amino acids, still more preferably of fewer than 10 amino acids.

TABLE 2
Kinetic parameters for 3 SCN5A clones.

| | | |
|---|---|---|
| hH1 | hH1a | hH1b |

Activation

| | | | |
|---|---|---|---|
| V_{1/2} (mV) | −44 ± 5 | −40 ± 7 | −43 ± 6 |
| Slope factor | 5 ± 1 | 5.5 ± 0.6 | 5.5 ± 0.7 |
| N | 10 | 7 | 9 |

Inactivation

| | | | |
|---|---|---|---|
| V_{1/2} (mV) | −95 ± 4.8* | −86 ± 5.8* | −90 ± 8.8 |
| N | 9 | 7 | 10 |

Recovery

| | | | |
|---|---|---|---|
| τ_{f} (ms) | 8 ± 5 | 5 ± 3 | 5 ± 2 |
| τₛ (ms) | 215 ± 101 | 279 ± 312 | 111 ± 52 |
| As | 0.31 ± 0.1 | 0.25 ± 0.1 | 0.24 ± 0.1 |
| N | 8 | 6 | 9 |

Decay (−30 mV)

| | | | |
|---|---|---|---|
| τ_{f} (ms) | 1.2 ± 0.4 | 1.5 ± 0.5 | 1.3 ± 0.5 |
| τₛ (ms) | 6.4 ± 5 | 6.0 ± 3 | 4.0 ± 1 |
| A_{f} | 0.71 ± 0.1 | 0.65 ± 0.1 | 0.56 ± 0.2 |
| N | 9 | 6 | 9 |

In a related aspect, the present invention also includes an immunogenic fragment of SEQ ID NO: 2 that contains at least one of amino acid 558 and amino acid 618 of SEQ ID NO: 2 and an antibody that binds specifically to such an immunogenic fragment. Such immunogenic fragments are used to generate an hH1b-specific antibody that can be used to detect or to isolate hH1b protein, or both. An hH1b-specific antibody has a higher affinity for hH1b protein than for either hH1- or hH1a protein. It is well within the ability of an artisan having ordinary skill in possession of the disclosed SEQ ID NO: 2 to make monoclonal or polyclonal antibodies against some or all of the polypeptide and to assess the specificity of the antibodies.

In another aspect, the present invention relates to an isolated nucleic acid containing a polynucleotide having an uninterrupted sequence that encodes a polypeptide of the invention as set forth above. An “isolated nucleic acid” has structure not identical to that of any naturally occurring nucleic acid or to that of any fragment of a naturally occurring genomic nucleic acid spanning more than three separate genes. The term therefore covers, for example, (a) a DNA that has the sequence of part of a naturally occurring genomic DNA molecule but which is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such as a cDNA or genomic DNA library. An isolated nucleic acid molecule can be modified or unmodified DNA or RNA, whether fully or partially single-stranded or double-stranded or even triple-stranded. A modified nucleic acid molecule can be chemically or enzymatically induced and can include so-called non-standard bases such as inosine.

In a related aspect, any nucleic acid of the present invention described above can be provided in a vector in a manner known to those skilled in the art. The vector can be a cloning vector or an expression vector. In an expression vector, the polypeptide-encoding polynucleotide is under the transcriptional control of one or more non-native expression control sequences which can include a promoter not natively found adjacent to the polynucleotide such that the encoded polypeptide can be produced when the vector is provided in a compatible host cell or in a cell-free transcription and translation system. Such cell-based and cell-free systems are well known to the skilled artisan. Cells comprising a vector containing a nucleic acid of the invention are themselves within the scope of the present invention. Also within the scope of the present invention is a host cell having the nucleic acid of the present invention integrated into its genome at a non-native site.

The present invention also includes an isolated nucleic acid molecule that contains a fragment of at least 20 contiguous nucleotides of SEQ ID NO: 1 or its complement wherein the fragment includes codon(s) encoding at least one of amino acids 558 and 618 and wherein the fragment hybridizes under stringent hybridization conditions to SEQ ID NO: 1 or its complement. Such a nucleic acid molecule can be used to detect the expression of hH1b in a cell. A stringent hybridization is, for example, hybridizing at 68° C. in 5×SSC/5×Denhardt's solution/1.0% SDS, and washing in 0.2×SSC/0.1% SDS at room temperature. Moderately stringent conditions include washing in 3×SSC at 42° C.

The present invention also enables a screening method for agents that can either inhibit or enhance sodium channel activities. In such a method, an agent is exposed to a cell line that expresses a functional hH1b and the agent's effect on hH1b activity is determined. hH1b activity can be measured in many ways, including but not limited to measuring a sodium current across the cell membrane, a sodium current kinetic activity, a membrane potential, or an intracellular sodium level. Also, a phenotype associated with over-expression of a sodium channel or absence of expression (e.g., in a transgenic knockout animal) can be monitored. In vitro, an effect on action potential can be measured after a channel of interest is transfected into suitable cells, such as cardiac cells. An arsenal of agents affecting the sodium channel activity are desired because many diseases and conditions, such as arrhythmias and Brugada syndrome, result from elevated or reduced sodium channel activity. Particularly in view of the understanding that various forms of the sodium channel α subunit differ functionally, it is important to evaluate the effects of every form that may be present in an individual. Indeed, one can tailor a suitable treatment to an individual after evaluating the form of α subunit present in that individual. Sodium channel activity means the open channel activity leading to a peak sodium current. Sodium channel activity is enhanced or inhibited when the open state probability is greater or less, and the peak current is higher or lower, respectively, than in the absence of a modulating agent.

Batteries of agents for screening are commercially available in the form of various chemical libraries including peptide libraries. Examples of such libraries include those from ASINEX (e.g., the Combined Wisdom Library of 24,000 manually synthesized organic molecules) and from CHEMBRIDGE CORPORATION (e.g., the DIVERSet™ library of 50,000 manually synthesized chemical compounds; the SCREEN-Set™ library of 24,000 manually synthesized chemical compounds; the CNS-Set™ library of 11,000 compounds; the Cherry-Pick™ library of up to 300,000 compounds). Once an agent having desired ability to increase or decrease activity of the sodium channel protein is identified, further iterations of the screen using one or more libraries of derivatives of that agent can be screened to identify agents having superior effects.

The above screening methods also enable one to determine the likelihood that an agent intended to be administered to a human or non-human subject will induce an undesired and unintended side effect, namely by altering the activity of cellular hH1b in a subject in which such alteration is not indicated. Any product of the invention described herein can be combined with one or more other reagent, buffer or the like in the form of a kit useful, e.g., for diagnostic or therapeutic purposes, in accord with the understanding of a skilled artisan.

The present invention is not intended to be limited to the foregoing, but rather to encompass all such variations and modifications as come within the scope of the appended claims. The invention will be more fully understood upon consideration of the following Examples which are, likewise, not intended to limit the scope of the invention.

### EXAMPLES

### Structural Differences Among Three Complete hNaᵥ1.5 Clones

We generated a novel, complete hNaᵥ1.5-encoding clone (SCN5A form hH1b) from human cardiac mRNA (Clontech®, Palo Alto, Calif.) using RT-PCR. About 3 kb of 5′ Naᵥ1.5 gene was cloned using 5′-GATGAGAAGATGGCAAACTTC C-3′ (SEQ ID NO: 3) and 5′-GCTCTGGATCCCCGGGGGTGCC-3′(SEQ ID NO: 4) primers. The 3 kb of 3′ Naᵥ1.5 gene was cloned with 5′-CACCCCCGGGGATCCAGAGC-3′ (SEQ ID NO: 5) and 5′-TTCAGTGTGTCCTGGCCAG-3′ (SEQ ID NO: 6) primers. RETRscript (Ambion®, Austin, Tex.) and pfu DNA polymerase were used to perform RT-PCR according to the protocol recommended by the manufacturer. PCR thermocycling involved one denaturation cycle at 94° C. for 1 min, 35 amplification cycles at 94° C. for 1 min, 50° C. for 1 min and 72° C. for 8 min, and one extension cycle at 72° C. for 20 min. The PCR products were cloned into pCR-BluntII-TOPO vector (Invitrogen®, Carlsbad, Calif.). The sequence of the hH1b gene was determined using thermostable polymerases and fluorescently labeled dideoxy terminators and automated DNA sequence analyzers at the University of Wisconsin Biotechnology Center. The two known hNaᵥ1.5-encoding clones (SCN5A forms hH1 and hH1a) were also re-sequenced completely.

### Kinetic Studies of the Three Complete hNaᵥ1.5 Clones

We measured macroscopic !Na of the three clones concurrently (on the same day) under identical conditions in the same experimental whole cell patch-clamp set-up. All techniques, protocols, and analysis techniques are standard and have been previously published (Nagatomo, et al., “Temperature dependence of early and late currents in human cardiac wild-type and long QT Δ KPQ Na⁺ channels,” Am.J.Physiol. (Heart44) 275, H2016-H2024 (1998), incorporated by reference herein as if set forth in its entirety). The pipette solution contained (in mM) 120 CsF, 15 CsCl, 2 EGTA, 5 HEPES and 5 NaCl (pH7.4 with CsOH). Data were recorded at room temperature. Peak I_{Na} and late I_{Na} were obtained after passive leak subtraction as described previously (Nagatomo, supra). Activation and inactivation data were fitted to a standard Boltzmann equation. Recovery and decay data were fitted to a two-exponential equation. Goodness of fit was determined both visually and by a sum of squares errors. One way ANOVA was performed to determine statistical significance among the three groups of mean data. Statistical significance was determined by a P value <0.05.

The 3 Naᵥ1.5 clones were introduced by transfection into the HEK293 host cell line (commercially available from ATCC) using Superfect (Qiagen®) according to the protocol recommended by the manufacturer. A Green Fluorescent Protein was co-transfected (at 1:10) as a marker to identify transfected cells. The vectors transfected included SCN5A form hH1 cloned into prcCMV vector (Invitrogen®, Carlsbad, Calif.), and forms hH1a and hH1b separately cloned into pcDNA3 vector (Invitrogen). HEK293 cells were cultured as previously described (Nagatomo et. al. 1998). The HEK 293 host cells were harvested 24 hours later to measure macroscopic I_{Na} current.

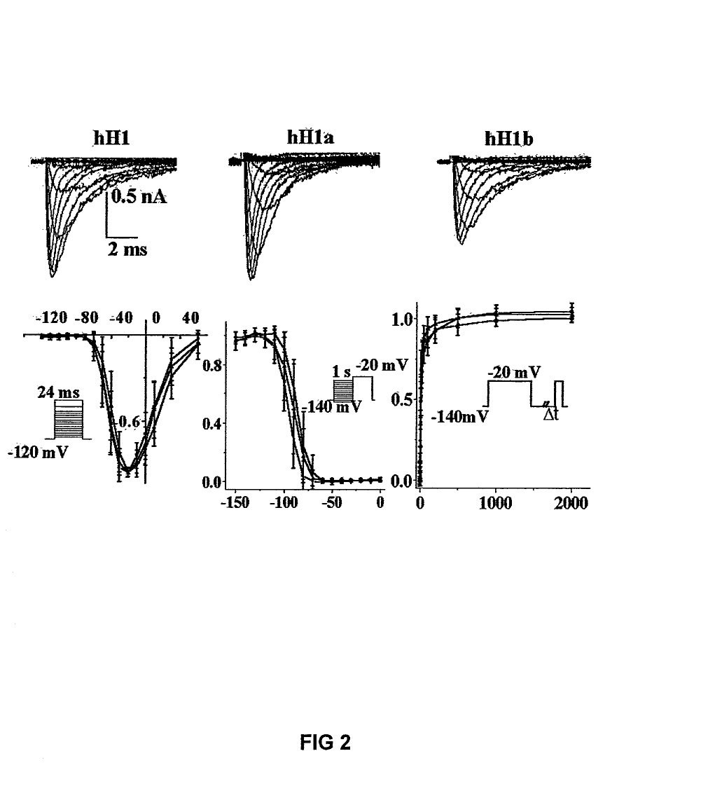

The three clones generally showed similar current time courses (FIG. 2a: representative traces were recorded with test potentials of −120 mV to +60 mV from a holding potential of −120 mV). Summary data for activation, steady-state inactivation, and recovery show minor differences (FIG. 2b Current-voltage relationship (left), “steady state” inactivation relationship (middle) and recovery from inactivation relationship (right) for I_{Na}: the y-axis represent normalized current; diagrams depicting the voltage clamp protocols are inset into each plot; peak I_{Na} was measured in response to the last depolarizing step and it was then normalized to the maximal peak I_{Na} found; solid symbols represent the mean data for between 6 to 10 experiments (see Table 2 for exact n numbers) with hH1 (square), hH1a (circle), and hH1b (triangle), and the bars represent SD). Although not identical, the kinetic parameters determined by the fitted lines (Table 2) were only statistically different for the midpoint of inactivation for hH1 (−95 mV) versus hH1a (−86 mV). Late I_{Na} measured at 240 ms after the start of the depolarization was no different among the three clones.

The three SCN5A clones, hH1, hH1a, and hH1b show minor differences in activation, inactivation, and recovery from inactivation kinetics. These parameters were obtained from fitting the individual experiments as in FIG. 2 to the appropriate model equations (Nagatomo, T. et al. (1998)). The fitted kinetic parameters from n experiments were averaged and are reported ±SD. For the Boltzmann fits (activation and inactivation), the parameters are V_{1/2} midpoint and slope factor. For the exponential fits (recovery and current decay), the parameters are τ_{f}—the fast time constant, τₛ—the slow time constant, Aₛ—the fractional amplitude of slow component, and A_{f}—the fractional amplitude of fast component. All parameters were analyzed by one-way ANOVA across the 3 clones, and statistically significant values are marked with an asterisk.

### Rescue of Expression for the M1766L Mutant by Using the hH1b Background

By site-directed mutagenesis, the M1766L arrhythmia mutation was engineered into all three clones, expressed in HEK cells, and studied by voltage clamp. Mutations were generated using the Excite® mutagenesis kit (Stratagene®). The mutagenesis method was based on the protocol recommended by the manufacturer. The M1766L mutation was created with 5′-TTCCTCATCGTGGTTAACCTGTACATTGCCATC-3 ′ (SEQ ID NO: 7) and 5′-GGAGATGATGATGTAGGTGG-3′ (SEQ ID NO: 8) primers. The histamine-to-arginine change at position 558 was generated with 5′-CGAGAGCCACCGCGCATCACTGCTG-3′ (SEQ ID NO: 9) and 5° CTCTCCCCCGCTGTGCTGTTTTC-3′ (SEQ ID NO: 10) primers. DNA was isolated and purified using a Qiagen® (Hilden, Germany) column and protocol.

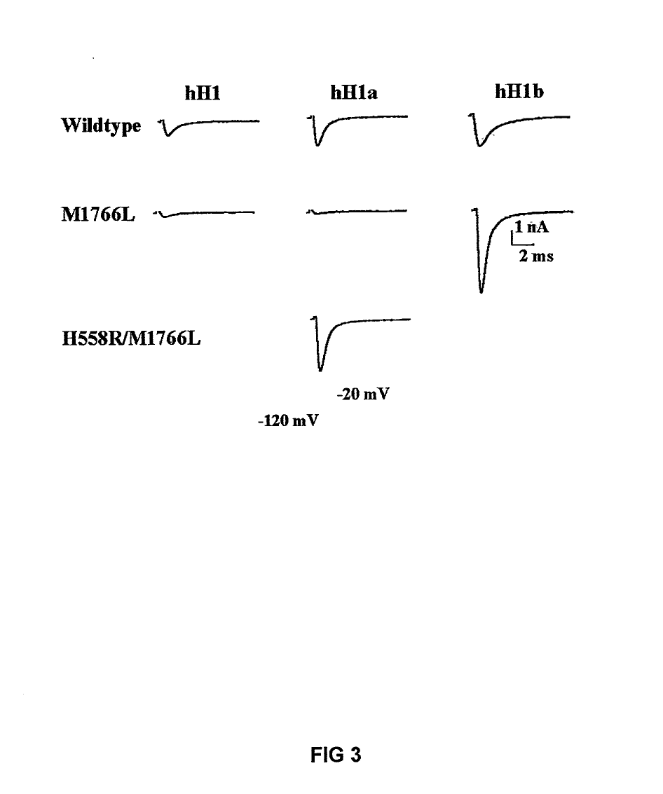

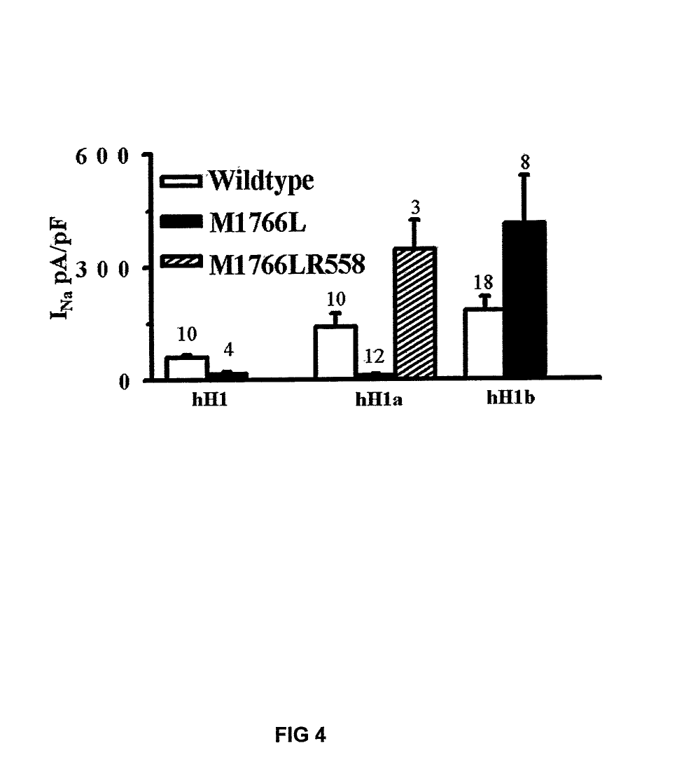

Examples Of I_{Na} traces show that the amount of current for the M1766L mutant channel was greatly reduced compared to wild type when expressed in hH1 and hH1a, but the current level was surprisingly large when expressed in hH1b (FIG. 3: Examples of I_{Na} traces (protocol inset) for each construct show that M1766L expressed very poorly in hH1 and hH1a, but expressed normally in hH1b; expression levels for the double mutant H558R/M1766L in hH1a were normal). Summary data of FIG. 4 show that for wild type, hH1b and hH1a clones generally expressed more I_{Na} than the hH1 clone. For wild type, but not mutant channels, the data here represent an underestimate of true current density because larger current densities were discarded to assure better voltage control for kinetic measurement. In general, however, wild type hH1 expressed at lower levels than hH1a and hH1b. Currents for M1766L in hH1b were dramatically greater than those for the mutant in hH1 and hH1a and were not affected by selection. I_{Na} were normalized to cell capacitance and mean values shown as a column with SD bar and the number of experiments indicated next to the bar. Without intending to be limited as to the theory underlying the invention, the applicants speculate that the H558R polymorphism underlies the restoration of expression in sodium channels carrying the M1766L mutation. Again using site-directed mutagenesis, we engineered the double mutation H558R/M1766L in the hH1a clone. Now, rather than the 97% reduction in current expression observed previously for M1766L in hH1a, the H558R/M1766L-hH1a mutant manifested a fully restored I_{Na} density (FIGS. 3 and 4).

We claim:

1. An isolated polypeptide comprising a sequence selected from the group consisting of SEQ ID NO: 2 and SEQ ID NO: 2 carrying a conservative substitution, deletion or rearrangement at one or more non-critical amino acid position.

2. An isolated polynucleotide that encodes a polypeptide comprising a sequence selected from the group consisting of SEQ ID NO: 2 and SEQ ID NO: 2 carrying a conservative substitution, deletion or rearrangement at one or more non-critical amino acid position.

3. The isolated polynucleotide of claim 2 comprising SEQ ID NO: 1.

4. The isolated polynucleotide of claim 2 operably linked to a non-native expression control sequence.

5. A cultured cell comprising a polynucleotide that encodes a polypeptide of claim 1.

6. The cultured cell of claim 5 comprising a polynucleotide that encodes a polypeptide that comprises a sequence selected from the group consisting of SEQ ID NO: 2 and SEQ ID NO: 2 carrying a conservative substitution, deletion or rearrangement at one or more non-critical amino acid position, the polynucleotide being operably linked to a non-native expression control.

7. An isolated nucleic acid comprising at least 20 contiguous nucleotides of SEQ ID NO: 1 including at least one of a codon that encodes amino acid 558 and a codon that encodes amino acid 618.

8. An antibody that binds specifically to an immunogenic fragment of SEQ ID NO: 2 that contains at least one of amino acid 558 and amino acid 618 of SEQ ID NO: 2

9. A method for producing a polypeptide comprising a sequence selected from the group consisting of SEQ ID NO: 2 and SEQ ID NO: 2 carrying a conservative substitution, deletion or rearrangement at one or more non-critical amino acid position, the method comprising the step of culturing the cell of claim 6 under conditions permitting expression of the polypeptide.

10. A method for identifying an agent that can alter the activity of a sodium channel relative to a standard, the method comprising the steps of:
providing a cultured cell comprising a polynucleotide that encodes a polypeptide comprising a sequence selected from the group consisting of SEQ ID NO: 2 and SEQ ID NO: 2 carrying a conservative substitution, deletion or rearrangement at one or more non-critical amino acid position, the polynucleotide being operably linked to a non-native expression control sequence;
producing the polypeptide in the cell;
exposing the cell to the agent; and
determining the sodium channel activity of the cell, an alteration in sodium channel activity being an indication that the agent is an activity-altering agent.

11. The method of claim 10, wherein the determining step comprises the step of measuring a parameter selected from the group consisting of a sodium current across a cellular membrane, a membrane potential, and an intracellular sodium level.

12. The method of claim 10, wherein the alteration is a decrease in sodium channel activity.

13. The method of claim 10, wherein the alteration is a increase in sodium channel activity.

14. A method for determining whether a biological sample contains an hH1b form of a sodium channel α subunit, the method comprising the steps of:
contacting the sample with an hH1b-specific antibody; and
determining whether the antibody specifically binds to a component in the sample, said binding being an indication that the sample contains the hH1b form of the sodium channel a subunit.

15. A method for determining whether a human or non-human subject is at risk for Long QT syndrome, the method comprising the step of:
determining whether the subject carries an hH1b form of an SCN5A gene.
